(19) United States
(12) Patent Application Publication
(10) Pub. No.: US 2026/0334835 A1
(43) Pub. Date: Oct. 29, 2026

(54) Sensors for Monitoring Bladder Pressure

(71) Applicant: Hollister Incorporated, Libertyville, IL (US)

(72) Inventors: Tadas S. Sileika, Lake Forest, IL (US); Nicole L. Dean, Antioch, IL (US); Sandra Stasiak, Mount Prospect, IL (US)

(21) Appl. No.: 19/110,759

(22) PCT Filed: Sep. 19, 2023
(86) PCT No.: PCT/US2023/074573

§ 371 Date: Mar. 11, 2025

Related U.S. Application Data
(60) Provisional application No. 63/408,563, filed on Sep. 21, 2022.

(51) Int. Cl. A61B 5/20; A61B 5/00

(52) U.S. Cl. CPC A61B 5/204; A61B 5/0059; A61B 5/205; A61B 5/6804; A61B 2560/0214; A61B 2562/0247; A61B 2562/0261; A61B 2562/164; A61B 2562/18

(57) ABSTRACT

A wearable skin contacting sensor assembly including a wearable fabric and a sensor configured to monitor a bladder condition. More particularly a wearable sensor including a piezoelectric material.

### FIELD OF THE INVENTION

This disclosure generally relates to a wearable sensor assembly for monitoring bladder conditions. More particularly, this disclosure relates to a sensor assembly wherein the sensor includes a piezoelectric material to measure bladder fullness and/or bladder pressure.

### BACKGROUND

Patients with spinal cord injuries (SCI) or other neurogenic bladder conditions typically need to empty their bladders by urinary catheterization, such as intermittent urinary catheterization. Catheterization may leave patients prone to urinary tract infections (UTIs). Patients may be prone to UTIs due to non-compliance with a regular catheterization schedule and/or not timely catheterizing when the bladder is full.

Aside from factors related to hygiene and the introduction of bacteria via catheterizing multiple times a day, there are risk factors for UTIs associated with altered bladder mechanics and compliance. For instance, detrusor overactivity, poor bladder compliance, and presence of vesicoureteral reflux (VUR) are associated with recurrent UTI. Additionally, hyperreflexic bladder with detrusor sphincter dyssynergia (DSD) is associated with higher incidence of UTI in SCI patients. Other risks include high intravesical pressures and disruption due to catheterization to the glycosaminoglycan layer (GAG), which is a barrier to bacterial invasion.

Currently, there are challenges with diagnosis and treatment of UTIs for SCI and neurogenic bladder patients. Properly diagnosing a patient with an SCI or other neurogenic bladder dysfunction may be difficult due to a variety of factors. For instance, neurogenic patients may demonstrate a variation in sensation, symptoms, and signs. Some patients may have an inability to sense and describe symptoms.

In certain instances, a urodynamic evaluation may be used to diagnose a patient with a UTI by measuring the function of the bladder. Urodynamic evaluations may identify hostile bladder conditions such as a high-pressure bladder or detrusor sphincter dyssynergia. Yet, obtaining a proper diagnosis via urodynamic evaluations may be challenging because they are awkward for the patient and unrealistic based on being performed in-office and with a limited data set. Additionally, follow-up evaluations can be too infrequent for some patients and conditions can exacerbate and be assessed too late.

Because UTIs provide significant health risks for patients with SCI or neurogenic bladder and are often not detected until it is too late, early detection and prevention of UTIs via monitoring presents opportunities to minimize such risks. Therefore, there is a need for improved methods, systems, and devices to monitor bladder conditions to, among other things, prevent UTIs.

### SUMMARY OF INVENTION

There are several aspects of the present subject matter which may be embodied separately or together in the subject matter claimed below. These aspects may be employed alone or in combination with other aspects of the subject matter described herein, and the description of these aspects together is not intended to preclude the use of these aspects separately or the claiming of such aspects separately or in different combination as set forth in the claims appended hereto.

In one aspect, a wearable skin contacting sensor assembly configured to monitor a bladder condition is provided. The sensor assembly includes a wearable patch and a sensor associated with the patch. The sensor includes a piezoelectric material. The piezoelectric material includes a current passing through the piezoelectric material, and the current is effected by stretching of a user's skin due to a change in volume of the user's bladder.

In another aspect, a system for monitoring bladder fullness or pressure is provided. The system includes a wearable skin contacting sensor assembly. The sensor assembly includes a wearable patch and a sensor associated with the patch. The sensor includes a piezoelectric material. The piezoelectric material includes a current passing through the piezoelectric material, and the current is effected by stretching of a user's skin due to a change in volume of the user's bladder. The system also includes a computing device configured to receive information from the sensor assembly.

In yet another aspect, a method for monitoring bladder fullness or pressure is provided. The method includes wearing a wearable skin contacting sensor assembly. The sensor assembly includes a wearable patch and a sensor associated with the patch. The sensor includes a piezoelectric material. The piezoelectric material includes a current passing through the piezoelectric material, and the current is effected by stretching of a user's skin due to a change in volume of the user's bladder. The method further includes transmitting sensor data to a computing device.

### BRIEF DESCRIPTION OF DRAWINGS

FIG. 1 is an enlarged schematic side view of an embodiment of a wearable sensor assembly;

FIG. 2 is a cross-sectional schematic top view of an embodiment of a wearable sensor;

FIG. 3 is an embodiment of a bladder monitoring system including an embodiment of a wearable sensor assembly and a computing device;

FIG. 4a is a schematic side view of an embodiment of a wearable sensor assembly when the bladder is empty; and

FIG. 4b is a schematic side view of the embodiment of the wearable sensor assembly of FIG. 4a when the bladder is filled.

### DETAILED DESCRIPTION

A more detailed description of the device in accordance with the present disclosure is set forth below. It should be understood that the description of the specific devices below is intended to be exemplary, and not exhaustive of all possible variations or applications. Thus, the scope of the disclosure is not intended to be limiting and should be understood to encompass variations or embodiments that would occur to persons of ordinary skill.

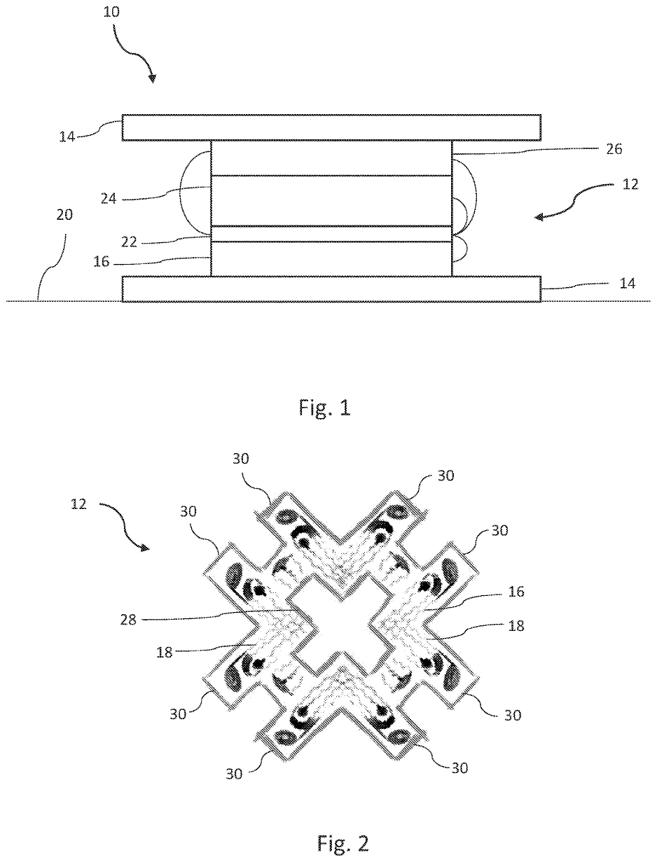

FIG. 1 shows a wearable skin-contacting sensor assembly 10. Wearable sensor assembly 10 includes a sensor 12 and a patch 14. The wearable sensor assembly 10 may be used to monitor a bladder condition, such as, but not limited to, the level of bladder fullness or the level of bladder pressure, blood flow in the area of the bladder, and the presence of various metabolic breakdown products, byproducts, and/or cellular components from various UTI-causing microorganisms. The sensor assembly 10 may be configured to monitor other bladder conditions without departing from the scope of the disclosure.

In some embodiments, the patch 14 may be, but is not limited to, a fabric such as a non-woven fabric, woven fabric, cotton fabric, synthetic fabric, and/or natural fabric. Other fabrics known in the art may be used without departing from the scope of the disclosure. The patch also may be made from a paper or a stretchable polymer material. In one embodiment, the patch 14 may be or may be incorporated into an article of clothing, such as male or female undergarments and/or accessories. The undergarment may be, but not limited to, boxers, briefs, panties, cheekies, thongs, merkins, jeans, exercise pants, yoga pants and shorts.

The sensor 12 may be attached to, embedded, incorporated into or within a compartment of the patch 14 such that when a user wears the sensor assembly 10, the sensor 12 is placed over the skin of the user. Depending on the construction of the wearable assembly 10, a surface of the patch 14 and/or a surface of the sensor 12 may be placed over the skin of a patient, with or without an intervening layer of adhesive (not shown) between the assembly 10 and the skin 20 of the patient. In one alternative the patch 14 and/or sensor 12 are located in the area right above the pubic bone, preferably in the area over the bladder. In one alternative, the sensor 12 may be sewn or embedded into the patch 14. In another embodiment, the sensor 12 may be attached or secured to the patch 14 with adhesive. In yet another embodiment, the patch 14, such as when the patch is an undergarment, may have a compartment to house the sensor 12. The compartment may be located in an area right above the pubic bone, and preferably in an area over the bladder. In yet another embodiment, the sensor 12 may be removeable from the compartment of the patch 14.

The sensor assembly 10 may be disposable or reusable. In some embodiments, the sensor 12 may be sealed with a polymer-based film. For example, the sensor 12 may be sealed with a thin polyolefin film. Other polymer-based films known in the art may be used without departing from the scope of the disclosure. By sealing the sensor 12 in a polymer-based film, the sensor assembly 10 may be washed without damaging the sensor 12 as necessary, for instance, after encountering bodily fluids. In one alternative, the sensor 12 sealed in a film may be removed from a compartment of the patch 14, washed and/or otherwise sanitized, and placed in another/different patch 14.

In an embodiment, sensor 12 may include a conductor, such as a piezoelectric material 16. In some embodiments, the piezoelectric material may be a matrix, such as a matrix of piezoelectric wires which may be strips of piezoelectric material or other suitable interconnected piezoelectric material. Other piezoelectric material known in the art may be used without departing from the scope of the disclosure. Current running through the piezoelectric material 16 may be used to determine bladder fullness or bladder pressure. The piezoelectric material 16 may serve to create a variable resistor. For example, the patch 14 and sensor 12 are placed over the skin in an area of the bladder of a user so that the piezoelectric material 16 (matrix of piezoelectric material) overlies the skin. When the skin 20 over the area of the bladder is stretched due to the bladder filling, a strain or force is placed on the piezoelectric material 16 which alters the current running through the piezoelectric material 16. The sensor 12 may sense the altered current which results in a changed data signal in the sensor 12. In one embodiment, the strain or force placed on the piezoelectric material 16 by the stretching of the skin, stretches or elongates the piezoelectric material 16, thereby resulting in the current change within the material 16.

In some embodiments, the sensor 12 may also include an integrated circuit controller 22. In an embodiment, the integrated circuit controller 22 may be coupled to the piezoelectric material 16. The integrated circuit controller 22 may be configured to measure the current or detect a change in the current running through the piezoelectric material 16.

The sensor 12 may also include a battery 24. Battery 24 may be coupled to the integrated circuit controller 22. In an embodiment, the battery 24 may be a rechargeable battery, such as a lithium battery, a nickel-metal hydride battery, a supercapacitor, or other means of electrical energy storage. Other batteries known in the art may be used without departing from the scope of the disclosure. The battery 24 may be recharged via near-field induction or a wired connection from an external electrical energy source, such as a battery pack, mobile electronic device, a mobile smart phone, or a wall outlet transformer. The battery 24 also may be reusable or used with multiple disposable devices. That is, battery 24 may be used with a first assembly and when the first assembly is spent, the battery 24 may be removed and used with a second assembly.

Additionally, the sensor 12 may include a data transmission device 26. Data transmission device 26 may be coupled to the integrated circuit controller 22. In an embodiment, the data transmission device 26 may transmit data from the sensor 12 to a computing device 34 wirelessly via wireless connections such as, near-field communication, Bluetooth, Low Energy Bluetooth, Zigbee, Z-wave, or other wireless connections known in the art. In another embodiment, the data transmission device 26 may transmit data from the sensor 12 to a computing device 34 using a wired connection such as via USB-C, Lightning, Firewire, or other means known in the art.

As also shown in FIG. 1, the sensor components may be arranged in a layered or stacked configuration. For instance, piezoelectric material 16 may be located closest to the skin 20. In some embodiments, the piezoelectric material 16 may be sewn directly into a weave of the patch 14. The integrated circuit controller 22, battery 24, and transmission device 26 may be stacked in any arrangement on top of the piezoelectric material 16. In another embodiment, the sensor 12 may include a diaphragm (not shown) abutting the piezoelectric material 16, between the piezoelectric material 16 and the skin 20. In this instance, as the skin 20 stretches, the skin 20 will apply a pressure to the diaphragm, which in turn will apply a pressure to the piezoelectric material 16, thus altering the current running through it.

FIG. 2 shows another embodiment of sensor 12. In the illustrated embodiment, the sensor 12 is shaped to have a perimeter of a block pound symbol (“#”). The sensor may be other shapes without departing from the scope of the disclosure. Sensor 12 may include a plurality of arms 30 and a sensor component hub 28 located in the middle of the sensor 12. In one embodiment, the sensor component hub 28 may be shaped like a plus sign (“+”). The sensor component hub 28 may include the integrated circuit controller 22, the battery 24, and/or the transmission device 26.

In one embodiment, the piezoelectric material 16 may extend throughout the entire shape of the sensor. In another embodiment, the piezoelectric material 16 may extend from the central sensor component hub 28 throughout the rest of the sensor 12. The piezoelectric material 16, for example piezo wires 18, may be localized in the arms of the sensor 12.

By arranging the integrated circuit controller 22, battery 24, and/or data transmission device 26 near the center of the sensor 12 and the piezoelectric material 16 throughout the sensor 12 or at the perimeter of the sensor 12, the sensor 12 may be thinner, thus increasing the sensitivity of the bending/flexing. This arrange may assist in allowing the piezoelectric material 16 to easily bend/flex as the bladder fills or is emptied.

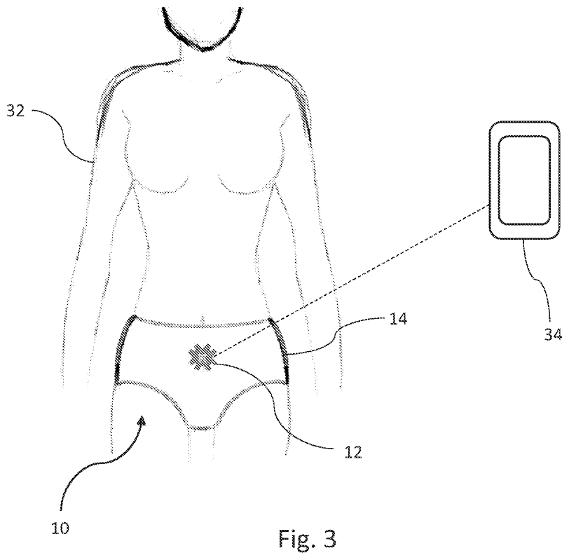

FIG. 3 shows a system including a sensor assembly 10 being worn by a user 32 and a computing device 34. The sensor assembly 10 may be one of the assemblies described herein. When worn, the sensor 12 of sensor assembly 10 is positioned to contact the skin just above the pubic bone, preferably over the bladder.

The sensor assembly 10 may communicate with a computing device 34 via a wireless or wired connection. The computing device 34 may be, for example, but not limited to, a mobile smart phone, a computer, or other smart devices. The computing device 34 contains an associated software application configured to at least receive, integrate, interpret, and/or analyze data transmitted from the sensor assembly 10. In some embodiments, the computing device 34 may be configured to display information received from the sensor 12.

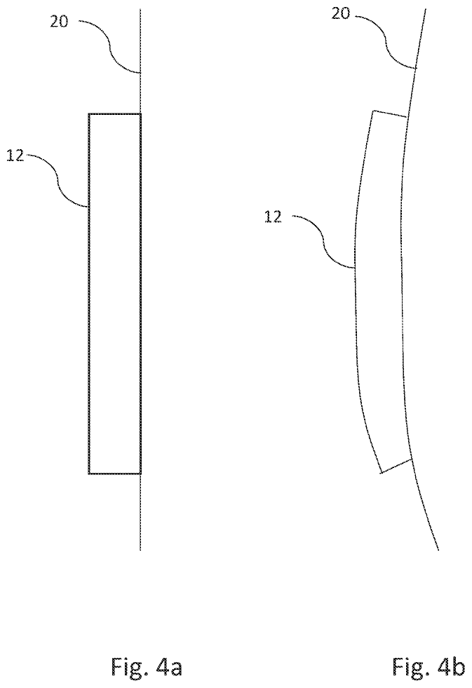

Upon initial use, the software application calibrates a baseline bladder fullness or pressure level when the bladder is empty. This is done after the user 34 has recently emptied their bladder. FIG. 4a shows an example of a sensor 12 overlying the skin 20 when the bladder is empty. Sensor 12 may not be completely flat when the bladder is empty because of the variations in body size from user to user, but it should be understood that the sensor 12 will conform to the curvature of the skin 20 above the bladder when the bladder is empty. The calibration accounts for regular movements during breathing, walking, and other daily motions over an established interval of time.

After calibration, the software application may interpret the baseline data of an empty bladder against ongoing signal collection. FIG. 4b shows an example of a sensor 12 overlying the skin 20 as the bladder fills. The current running through the piezoelectric material 16 will alter as the skin 20 stretches and flexes the piezoelectric material 16. The change in current may be collected by the sensor 12 during ongoing signal collection. If the signal reaches a critical level, which can be specified by a clinician or from repeat data collected for a demographic or a given user, the patient is notified by the application to empty the bladder. Furthermore, the application may couple with a variety of health tracking devices, for instance, smart watches, such as but not limited to a FitBit, Apple Watch, Garmin Watch, and other smart watches to notify the user when it is time to empty the bladder.

In some embodiments the sensor 12 may be augmented or replaced with a near-infrared spectroscopy (NIRS) system. The system may include infrared and near infrared light emitting diodes (LEDs) to measure blood flow in the area of the bladder. Additionally, the sensor 12 may also include a photoelectric sensor or charge coupled device to collect reflected signals emitted from the LEDs. In one embodiment, the system may monitor hemodynamic changes around the bladder to detect bladder fullness and/or bladder pressure. Hemodynamic changes may be associated with tissue chromophore levels, including, but not limited to, oxyhemoglobin and deoxyhemoglobin.

As motion can introduce artifact to the NIRS readings, a baseline calibration may be performed to account for regular daily motions of the user, including but not limited to sleeping, ambulating, and eating. Calibration of an empty or full bladder can be done through a cystometry analysis done at a clinician's office or may be compared against baseline cystometry data from similar cohorts and demographics. The calibration allows to establish a reference value table of what coincides with an empty and a full bladder. Signal readings compared to a baseline reference may be used to appropriately push notification to a computing device to alert the user to empty the bladder as needed.

In yet another embodiment, the NIRS system may be utilized to detect various metabolic breakdown products, byproducts, and/or cellular components from various UTI-causing microorganisms. In one embodiment, the metabolic products, byproducts, and/or cellular components may be ammonia (as generated by Proteus mirabilis) and/or endotoxins, for example, Escherichia coli. Other products, byproducts, and/or cellular components known in the art may be monitored for without departing from the scope of the disclosure.

As with bladder fullness, baseline data can be collected from the user in a healthy state. Baseline data may be determined by a clinician or by utilizing data from baseline demographics. If suspected UTI components are detected by the system, the application may notify the user, or potentially the clinician, if the computing device is connected to the internet. Additionally, the application may instruct the user of potential interventions, such as antibiotic usage or implementation of non-clinical approaches. Furthermore, the application may provide for input from the user into what means of UTI mitigation the user undertook, integrating with a database that allows for evaluation of successful interventions (via artificial intelligence, for example).

It will be understood that the embodiments and examples described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A wearable skin contacting sensor assembly configured to monitor a bladder condition comprising:
a wearable patch; and
a sensor associated with the patch, the sensor comprising a piezoelectric material, wherein a current passing through the piezoelectric material is effected by stretching of a user's skin due to a change in volume of the user's bladder.

2. The sensor assembly of claim 1, wherein the piezoelectric material comprises a matrix of the piezoelectric material.

3. The sensor assembly of claim 1, wherein the piezoelectric material extends throughout the shape of the sensor.

4. The sensor assembly of claim 1, wherein the sensor comprises an integrated circuit controller configured to measure the current passing through the piezoelectric material.

5. The sensor assembly of claim 1, wherein the sensor comprises a power source.

6. The sensor assembly of claim 5 wherein the power source is a rechargeable battery.

7. The sensor assembly of claim 1, wherein the sensor comprises a data transmission device.

8. The sensor assembly of claim 1, wherein the sensor comprises a diaphragm located adjacent to the piezoelectric material between the user's skin and the piezoelectric material.

9. The sensor assembly of claim 1, wherein the sensor comprises an infrared or near infrared light emitting diode and a photoelectric sensor.

10. The sensor assembly of claim 1, wherein the sensor is sealed with a polymer-based film.

11. The sensor assembly of claim 10, wherein the polymer-based film is a polyolefin film.

12. The sensor assembly of claim 1, wherein the sensor is embedded into the patch.

13. The sensor assembly of claim 1, wherein the sensor is removably attached to the patch.

14. The sensor assembly of claim 1, wherein the patch is non-woven fabric, woven fabric, cotton fabric, synthetic fabric, or natural fabric.

15. The sensor assembly of claim 1, wherein the patch comprises an article of clothing.

16. The sensor assembly of claim 1, wherein the sensor is positioned over the bladder of a user when the sensor assembly is worn.

17. A system for monitoring bladder pressure comprising:
a wearable skin contacting sensor assembly, wherein the sensor assembly comprises
a wearable patch;
a sensor associated with the wearable patch, the sensor comprising a piezoelectric material, wherein a current passing through the piezoelectric material is effected by stretching of a user's skin due to a change in volume of the user's bladder; and
a computing device configured to receive information from the sensor assembly.

18. The system of claim 17, wherein the piezoelectric material comprises a matrix of the piezoelectric material.

19. The system of claim 17, wherein the piezoelectric material extends throughout the shape of the sensor.

20-38. (canceled)

39. A method for monitoring bladder pressure comprising:
wearing a wearable skin contacting sensor assembly, wherein the sensor assembly comprises
a wearable patch and a sensor associated with the patch, the sensor comprising a piezoelectric material, wherein a current passing through the piezoelectric material is effected by stretching of a user's skin due to a change in volume of the user's bladder; and
transmitting sensor data to a computing device.

40-65. (canceled)
